(19) United States
(12) Patent Application Publication
(10) Pub. No.: US 2025/0084016 A1
(43) Pub. Date: Mar. 13, 2025

(54) LIQUID ORGANIC HYDROGEN CARRIER PROCESS AND CATALYST SYSTEM

(71) Applicant: UOP LLC, Des Plaines, IL (US)

(72) Inventors: Nirlipt Mahapatra, Haryana (IN); Priyank Tiwari, Haryana (IN); Srinivasan Ramanujam, Haryana (IN); Priyesh Jayendrakumar Jani, Haryana (IN); Gautam Pandey, Haryana (IN); Pankaj Kumar Singh, Buffalo Grove, IL (US); Raymond S. Chan, Ottershaw (GB); Bryan J. Egolf, Crystal Lake, IL (US); Elie J. Fayad, Munster, IN (US); David S. Lafyatis, Schaumburg, IL (US); Clayton C. Sadler, Arlington Heights, IL (US)

(21) Appl. No.: 18/744,983

(22) Filed: Jun. 17, 2024

Related U.S. Application Data
(60) Provisional application No. 63/581,696, filed on Sep. 11, 2023.

(51) Int. Cl. C07C 5/10; B01D 53/22; C01B 3/50; C01B 3/56; C07C 5/32

(52) U.S. Cl. CPC C07C 5/10; B01D 53/22; C01B 3/501; C01B 3/56; C07C 5/322; B01D 2256/16; C07C 2521/04

(57) ABSTRACT

Processes for hydrogenating toluene to methylcyclohexane (MCH) and dehydrogenating MCH to toluene with minimal to no by-products, thereby ensuring minimal loss of hydrogen are described. MCH acts as a liquid organic hydrogen carrier, and it can be transferred in storage vessels and/or pipelines for several thousands of miles to the final destination with very minimal to no degradation.

### RELATED APPLICATIONS

This application claims priority to U.S. Provisional Patent Application Ser. No. 63/581,696, filed on Sep. 11, 2023, the entirety of which is incorporated herein by reference.

### BACKGROUND

Hydrogen is expected to have significant growth potential because it is a clean-burning fuel. Aggressive carbon emission reduction targets and rising carbon penalties for 2030-2050 to meet the Paris Climate Agreement are anticipated to drive a hydrogen-based economy in the near future.

However, hydrogen production processes based on steam reforming, autothermal reforming, partial oxidation, or gasification of hydrocarbon or carbonaceous feedstocks are significant emitters of CO₂. Government regulations and societal pressures are increasingly taxing or penalizing CO₂ emissions or incentivizing CO₂ capture. Consequently, there is significant interest in lowering the cost of hydrogen production using these processes and recovering the byproduct CO₂ for subsequent geological sequestration. Hydrogen from solar, wind, and water (Green Hydrogen), which does not involve the production of CO₂, could meet projected global energy demand in the future and play a vital role in reducing global warming. The recently renewed interest in alternative energy sources and energy carriers opens up new prospects for this process to be applied as a feed system for fuel cells, power generation and many more applications.

There exists a huge regional disparity in the cost of production of hydrogen. A number of technologies have been developed for transporting hydrogen, including NH₃, liquid H₂, and liquid organic hydrogen carrier (LOHC) to address this disparity. Toluene-Methylcyclohexane (MCH) is expected to be a significant player in the LOHC space considering numerous advantages, such as easy integration with existing fuel sector supply chain and distribution network, utilization in idle refinery assets, flexibility for co-processing, and higher relative handling safety.

LOHC involves the reversible dehydrogenation reaction of methylcyclohexane (MCH) to produce toluene (TOL) and hydrogen (through the so called MTH cycle). It has been proposed as a solution for the storage, transportation, and distribution of hydrogen produced from renewable energy sources. For power generation, the hydrogen from this process is usually compressed for the downstream power generation unit. Usually, the purity requirement for power generation unit is very stringent. Due to the relatively high cost associated with the green hydrogen production, it is necessary to recover almost all hydrogen.

Accordingly, it would be desirable to have more effective and efficient ways to purify and transport hydrogen and in particular hydrogen produced from a renewable resource.

### BRIEF DESCRIPTION OF THE DRAWINGS

FIG. 1 is an illustration of one embodiment of a toluene saturation process according to the present invention.

FIG. 2 is an illustration of one embodiment of a methylcyclohexane dehydrogenation process according to the present invention.

FIG. 3 is a graph showing the single pass yield of methylcyclohexane in a reactor as a function of temperature.

### DESCRIPTION

The process involves a method of hydrogenating toluene to methylcyclohexane (MCH) and dehydrogenating MCH to toluene with minimal to no by-products, thereby ensuring minimal loss of hydrogen (green or blue hydrogen). MCH acts as a liquid organic hydrogen carrier, and it can be transferred in storage vessels and/or pipelines for several thousands of miles to the final destination with very minimal to no degradation. This invention helps address the supply and demand gap of blue and green H₂ as well as the huge differential cost of production between regions. It involves the novel integration of toluene saturation reaction system with a H₂ separation system. The goal is to maximize the pressure without changing the vapor to a liquid to increase the reaction driving force, minimize capital costs, and enable the separator to absorb trace byproducts so it can be removed in the stabilizer. For this catalytic reaction system, vapor phase needs to be maintained. Therefore, the pressure can only be increased up to the dew point of the reactor system vapor. This in turn results in the small amount of non-selective trace byproducts produced in the reactor to not be absorbed with the reactor effluent liquid. Simulations showed trace byproducts building up in the reactor circuit leading to high capital and energy costs. The addition of a H₂ separation system on a slip stream from the separator vent gas enabled the reduction in the capital and operating costs of the reactor section, while also producing a high purity H₂ stream. An unexpected benefit was that it also enabled the use of H₂ in the stabilizer off gas, therefore reducing the amount of makeup hydrogen needed. The makeup gas compressor can also be used to compress the permeate, avoiding the need for an additional permeate compressor. The process involves the hydrogenation of toluene to methylcyclohexane at a first location, transfer of the methylcyclohexane to a second location, and dehydrogenation of the methylcyclohexane to toluene and hydrogen at the second location.

Although toluene saturation catalysts have very high selectivity (e.g., greater than 99.9%), trace by-products (such as C₁-C₃ hydrocarbons) may be formed by metal catalyzed dealkylation. Byproduct formation can vary depending on the presence of contaminants in the feed; it can also vary between the start of the run and the end of the run. Considering the limited sponging capability of methylcyclohexane at optimized separator pressures, trace by-products would build up in the loop leading to several problems, such as higher compressor duty, hydrogen losses and low technology life. Lower H₂ partial pressure at constant separator pressure also leads to lower conversion than required resulting in unconverted toluene being present in the MCH shipment.

One solution is to take a slip stream from the separator overhead and send it to a one- or two-stage membrane or sponge oil system. The permeate can be sent to the makeup gas compressor (MGC) suction along with a fraction of the stabilizer off-gas. This allows maximization of H₂ recovery.

In addition, medium pressure steam generated from the heat of reaction of the first, intermediate, and second (and any additional) reactors is superheated using the heat of reaction of the intermediate, second (or subsequent) reactor to make it more suitable for power generation and export. The process can incorporate heat integration with speciality heaters and pressure recovery turbines (PRTs) in order to derive the maximum power from the process. This is especially beneficial when green H₂ is produced in a remote location. The power generated can be used to run the unit, and it can also be exported/supplied to integrated hydrogen generation units, such as electrolyzers.

The hydrogenation reaction section comprises two or more hydrogenation reactors. A hydrocarbon stream comprising toluene is split into two or more streams. The first toluene stream is sent to the first hydrogenation reactor where the toluene is hydrogenated into methylcyclohexane. The first saturated reactor effluent stream is sent to a second hydrogenation reactor along with the second hydrocarbon stream where toluene is hydrogenated to produce additional methylcyclohexane. There can be one or more additional hydrogenation reactors between the first and second hydrogenation reactors. The second saturated reactor effluent is sent to a polishing reactor for additional reaction of toluene to methylcyclohexane.

Hydrogen is present in the first and second hydrogenation reactors. It can be added to the hydrocarbon feed stream, first hydrocarbon stream, the first hydrogenation reactor, the second hydrocarbon stream, the second hydrogenation reactor, or combinations thereof. No hydrogen is added to the second saturated reactor effluent or the polishing reactor.

The polishing reactor effluent is separated in a high-pressure separator into a liquid stream comprising methylcyclohexane and a vapor stream comprising hydrogen. At least a portion of the hydrogen in the first and/or second hydrogenation reactors (whether added to the reactor or mixed with the hydrocarbon feed stream and/or the first and/or second hydrocarbon streams) comes from the vapor stream.

The liquid stream is split into an optional recycle hydrocarbon stream and a second liquid stream. The optional recycle hydrocarbon stream can optionally be recycled to the first and/or second hydrogenation reactors (whether added directly to the first and/or second hydrogenation reactors or by mixing with the hydrocarbon feed stream and/or the first and/or second hydrocarbon streams).

The second liquid stream is sent to a stabilizer column to remove dissolved gases to form a product stream comprising methylcyclohexane. The second liquid stream can be sent through a pressure recovery turbine before it is stabilized to generate power and the power can be utilized in the hydrogenation process or exported to a second process.

Part of the off-gas stream from the stabilizer overhead stream can be recycled to a make-up gas compressor suction.

A portion of the vapor stream is passed through a membrane or sponge oil system forming a hydrogen rich stream and a byproduct rich reject stream. The hydrogen rich stream is compressed in one or more compressors and sent to the first and/or second hydrogenation reactors and/or mixed with the hydrocarbon feed stream, and/or the first and/or second hydrocarbon streams. When a membrane is used, the hydrogen rich stream is the hydrogen rich permeate stream, and the byproduct reject stream is the byproduct rich retentate stream. The portion of the vapor stream sent through the membrane or sponge oil system is typically in the range of 0.01 to 35%, or 0.01 to 30%, or 0.01 to 25%, or 0.01 to 20%, or 0.05 to 35%, or 0.05 to 30%, or 0.05 to 25%, or 0.05 to 20%, or of 1 to 35%, or 1 to 30%, or 1 to 25%, or 1 to 20%, or 5 to 35%, or 5 to 30%, or 5 to 25%, or 5 to 20%.

The hydrocarbon feed stream can be pre-heated using the heat of reaction from the first hydrogenation reactor and/or the second hydrogenation reactor and/or the intermediate hydrogenation reactor.

A steam stream can be generated from the heat of reaction of one of the hydrogenation reactors, and the steam stream can be superheated using the heat of reaction of another hydrogenation reactor. The superheated steam stream can be used to generate power. The power can be used in the hydrogenation process or exported to another process. The steam stream can have a pressure in the range of 350 kPa (g) to 2500 kPa (g) (3.5 barg to 25 barg).

The hydrocarbon feed stream, the hydrogen, or both can be treated in an adsorbent bed to remove contaminants.

Any suitable hydrogenation catalysts may be used as the first, intermediate, second, and polishing hydrogenation catalysts. The first, intermediate, second, and/or polishing catalysts can be the same or different. The hydrogenation catalyst should have high selectivity and a low rate of coke lay down. Suitable hydrogenation catalysts for the first, intermediate, second, and/or polishing hydrogenation catalyst include, but are not limited to, a metal of Group VIII of the Periodic Table and optionally a metal of Group I of the Periodic Table. Suitable hydrogenation catalysts for the first, intermediate, second, and/or polishing hydrogenation catalyst also include, but are not limited to, 0.05 wt % to 30 wt % of a metal of Group VIII of the Periodic Table and optionally 0.1 wt % to 3 wt % of a metal of Group I of the Periodic Table.

When an additional hydrogenation reactor is present between the first and second hydrogenation reactors, the hydrocarbon feed stream is split into at least the first and second hydrocarbon streams and a third hydrocarbon stream. The first saturated effluent stream and the third hydrocarbon stream are sent to the intermediate hydrogenation reactor in the presence of hydrogen and an intermediate hydrogenation catalyst to form an intermediate saturated effluent stream comprising additional methylcyclohexane. The intermediate saturated effluent stream is sent to the second hydrogenation reactor.

The process mitigates the problems associated with the production of lights ends. There is a slip stream from the recycle gas loop stream, typically about 0.01-35%, that is sent through a membrane or sponge oil system. Additionally, there is off-gas recycle to the make-up gas suction.

Typical operating pressures for the first, second, intermediate, and polishing reactors are in the range of 1034-6895 kPa (150 to 1000 psig), or 2068-3447 kPa (300 to 500 psig). Typical inlet temperatures for all of the reactors are in the range of 204-232° C. (400-450° F.). Typical outlet temperatures for the first, second, and intermediate reactors are in the range of 316-371° C. (600-700° F.). Typical outlet temperatures for the polishing reactor are in the range of 218-260° C. (425-500° F.)

The polishing reactor may have an inlet temperature at least 20° C. higher than a dew point of the second or intermediate reactor effluent stream.

The product stream comprising the methylcyclohexane is then transferred to a second location. The methylcyclohexane feed from the storage tanks which may not be completely dry or properly nitrogen-blanketed may be treated in an oxygen stripper before being routed to the dehydrogenation reactor section. The methylcyclohexane feed is mixed with a recycle methylcyclohexane stream from a deisoheptanizer column. The combined methylcyclohexane feed is mixed with recycle hydrogen and then preheated by exchange with the reactor effluent in a combined feed exchanger (CFE). The combined feed is then raised to the reaction temperature in the convection section of a charge heater and sent to the dehydrogenation reactor section comprising one or more dehydrogenation reactor(s). The combined feed passes through the dehydrogenation reactor(s). The dehydrogenation reactor(s) are typically radial flow reactors. Interheaters are used to raise the reactor effluent back to the desired reactor inlet temperature for the next dehydrogenation reactor. The effluent from the last dehydrogenation reactor is cooled in the combined feed exchanger and the product condenser before passing to the separator.

The separator vapor is compressed by the recycle gas compressor and split into net gas and recycle gas. The recycle gas is sent back to the combined feed exchanger to be mixed with the feed. The net gas is the hydrogen gas product stream and is sent to the hydrogen gas compression section. Toluene-rich liquid from the separator is pumped to the stabilizer column.

The hydrogen gas compression section comprises one or more hydrogen gas compressor(s) which provide sufficient pressure to meet the hydrogen purity requirements of the user. The hydrogen purity increases through each stage of compression. In addition, there is some liquid product recovered that is sent to the stabilizer column.

The toluene-rich liquid from the separator in dehydrogenation reactor section and the liquid product recovered from hydrogen gas compression section are combined and sent to the stabilizer column to recover the toluene. In the stabilizer column, hydrogen and any trace byproducts are removed from the toluene-rich feed as a stabilizer column overhead offgas stream. The offgas stream can be sent to the refinery or used as fuel gas, for example. The stabilizer column bottoms stream may be used to heat the feed to the stabilizer column, It may be sent to the deisoheptanizer column for further fractionation or to storage, depending on the required toluene specification, for shipping.

Any suitable dehydrogenation catalyst that can achieve a selectivity in the dehydrogenation of methylcyclohexane to toluene and hydrogen in excess of 99.8% can be used. Suitable dehydrogenation catalysts include, but are not limited to, Al₂O₃, a noble metal, and an alkali or alkaline earth metal. Suitable noble metals include, but are not limited to, Pt, Pd, Rh, Ru, Re, Ir, Au, Os, Ag, or combinations thereof. Suitable alkali or alkaline earth metals include, but are not limited to, Na, Cs, K, Rb, Fr, Li, Be, Sr, Ba, Ca, Mg, Ra, or combinations thereof.

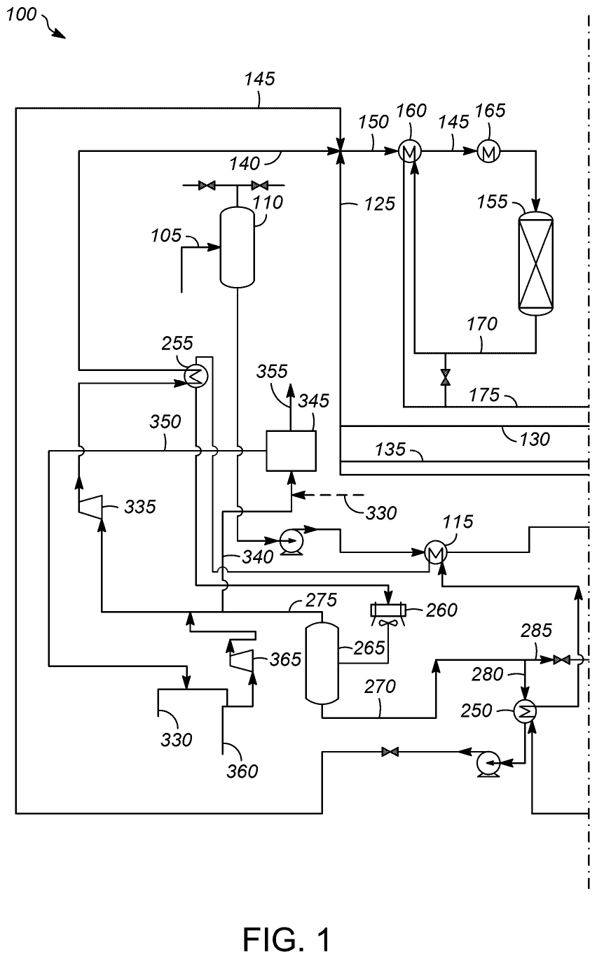

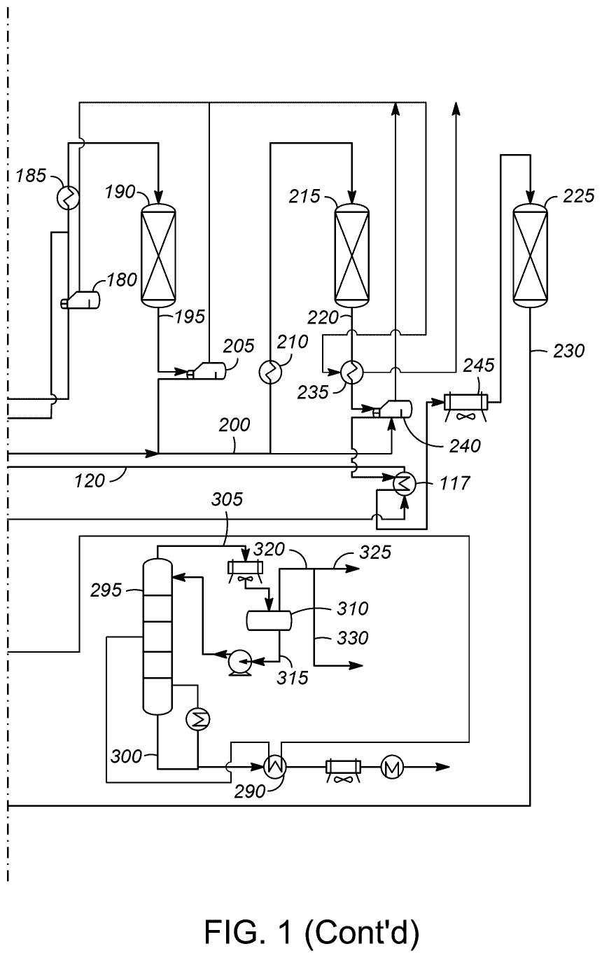

FIG. 1 illustrates one embodiment of the toluene hydrogenation process 100.

The hydrocarbon feed stream 105 from a storage tank (not shown) can be sent to a feed surge drum 110. It can be preheated in one or more feed preheaters 115, 117. The preheated feed stream 120 is split into 3 hydrocarbon streams 125, 130, 135.

The first hydrocarbon stream 125 is mixed with a hydrogen recycle stream 140 and a hydrocarbon recycle stream 145. Alternatively, the hydrogen recycle stream 140 and/or the optional hydrocarbon recycle stream could be introduced directly into the first hydrogenation reactor 155.

The combined feed stream 150 can optionally be further heated in a heat exchanger 160 by the first saturated effluent stream 170 and/or a heater 165 to obtain the desired initial temperature for the hydrogenation reaction (e.g., about 425° F. (218° C.)).

The combined feed stream 150 is sent to the first hydrogenation reactor 155 of the hydrogenation reaction section where the toluene is converted to methylcyclohexane. The first saturated effluent stream 170 comprising the converted methylcyclohexane exits the first hydrogenation reactor 155 at a temperature of about 650° F. (343° C.).

The first saturated effluent stream 170 is combined with the intermediate hydrocarbon stream 130 comprising toluene forming a combined intermediate stream 175.

The first saturated effluent stream 170 and/or the combined intermediate stream 175 is cooled by one or more of heat exchanger 160, the first steam generator 180, and/or the first feed air cooler 185 to the desired temperature for the intermediate hydrogenation reactor 190 (e.g., about 425° F. (218° C.)).

The combined intermediate stream 175 is sent to the intermediate hydrogenation reactor 190. where toluene is converted to additional methylcyclohexane. The intermediate saturated effluent stream 195 exits the exits the intermediate hydrogenation reactor 190 at a temperature of about 650° F. (343° C.).

The intermediate saturated effluent stream 195 is combined with the second hydrocarbon stream 135 to form second combined stream 200.

The intermediate saturated effluent stream 195 and/or the second combined stream 200 is cooled by one or more of the second steam generator 205, and/or the second feed air cooler 210 to the desired temperature for the second hydrogenation reactor 215 (e.g., about 425° F. (218° C.)).

The second combined stream 200 is sent to the second hydrogenation reactor 215. where toluene is converted to additional methylcyclohexane. The second saturated effluent stream 220 exits the exits the second hydrogenation reactor 215 at a temperature of about 650° F. (343° C.).

The second saturated effluent stream 220 is sent to polishing hydrogenation reactor 225 where any remaining toluene is converted to additional methylcyclohexane. The polishing saturated effluent stream 230 exits the exits the polishing hydrogenation reactor 225 at a temperature of about 460° F. (238° C.).

The second saturated effluent stream 220 is cooled by one or more of the steam superheater 235, the third steam generator 240, the second feed preheater 117, and/or the third reactor effluent cooler 245 to the desired temperature for the polishing hydrogenation reactor 225 (e.g., about 425° F. (218° C.)).

The polishing saturated effluent stream 230 is sent to separator liquid-reactor effluent exchanger 250, first feed preheater 115, recycle gas preheater 255, product condenser 260, and separator 265 where it is separated into separator liquid stream 270 comprising methylcyclohexane and separator overhead vapor stream 275 comprising hydrogen and light ends.

A portion 280 of separator liquid stream 270 is sent to separator liquid-reactor effluent exchanger 250 forming hydrocarbon recycle stream 145.

The remainder 285 of the separator liquid stream 270 is sent to stabilizer feed-bottoms exchanger 290 and stabilizer column 295. The stabilizer bottoms stream 300 comprising methylcyclohexane is sent to the stabilizer feed-bottoms exchanger 290 and sent to storage.

The stabilizer overhead stream 305 is separated in separator 310 into reflux stream 315 and offgas stream 320. Reflux stream 315 is returned to the stabilizer column 295. Offgas stream 320 is split into offgas purge stream 325 and offgas recycle stream 330.

The separator overhead vapor stream 275 is compressed in recycle gas compressor 335 and sent to recycle gas preheater 255 forming the hydrogen recycle stream 140.

Optionally, a slip stream 340 of the separator overhead vapor stream 275 is sent to a membrane separator and/or a sponge oil system 345. The membrane separator 345 can include one or more membranes (e.g., one, two, three, etc.). The slip stream 340 is separated into a hydrogen rich permeate stream 350 and a methane rich retentate stream 355. The retentate stream can be sent to a vent header.

The hydrogen rich permeate stream 350 is combined with the offgas recycle stream 330 from the stabilizer column 295 and makeup hydrogen gas 360, compressed in the makeup gas compressor 365, and combined with the separator overhead vapor stream 275. Alternatively, the offgas recycle stream 330 can be compressed and sent to the inlet of the membrane separator and/or a sponge oil system 345

The stored methylcyclohexane of the stabilizer bottoms stream 300 is transported to a second location for dehydrogenation to toluene and hydrogen.

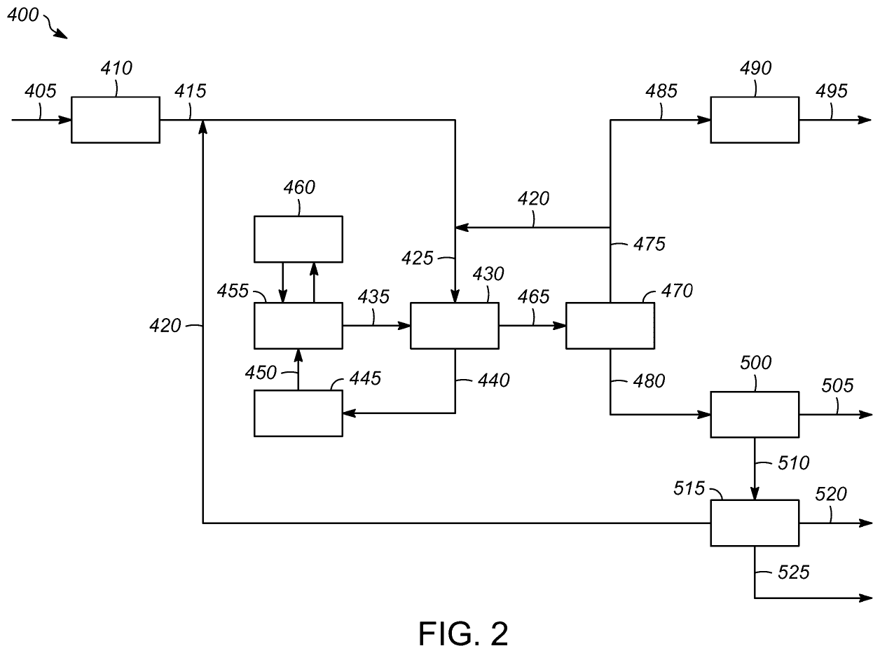

FIG. 2 illustrates one embodiment of a dehydrogenation process 400.

The methylcyclohexane feed stream 405 may be sent to an optional oxygen stripper 410 to remove oxygen. The deoxygenated methylcyclohexane 415 is combined with a recycle methylcyclohexane stream 420 and a recycle hydrogen stream 420 forming a combined feed stream 425. The combined feed stream 425 is sent to a combined feed exchanger 430 to preheat the combined feed stream 425 by heat exchange with the dehydrogenation reactor effluent 435. The preheated combined feed stream 440 is further heated in a charge heater 445 to the reactor temperature. The heated combined feed stream 450 is sent to the dehydrogenation reaction section 455 comprising one or more dehydrogenation reactor(s). One or more interheater(s) 460 raise the reactor effluent back to the desired reactor inlet temperature between dehydrogenation reactor(s).

The dehydrogenation reactor effluent 435 from the last dehydrogenation reactor is cooled in the combined feed exchanger 430. The cooled reactor effluent stream 465 is sent to a gas/liquid separator 470 where it is separated into a separator vapor stream 475 comprising hydrogen and a separator liquid stream 480 comprising toluene.

The recycle hydrogen stream 420 is split from the separator vapor stream 475, and the remainder 485 is sent to a hydrogen compressor 490 forming the hydrogen product stream 495.

The separator liquid stream 480 is sent to a stabilizer column 500 (as well as any liquid from hydrogen compressor 490). The overhead stream 505 from the stabilizer column 500 comprises hydrogen and light ends which can be sent to the refinery or used as fuel gas. The stabilizer bottoms stream 510 is sent to a deisoheptanizer column for further fractionation into a C6/C7 stream 520 comprising hexane and heptane, the recycle methylcyclohexane stream 420, and the toluene product stream 525.

### EXAMPLES

### Example 1—Hydrogenation

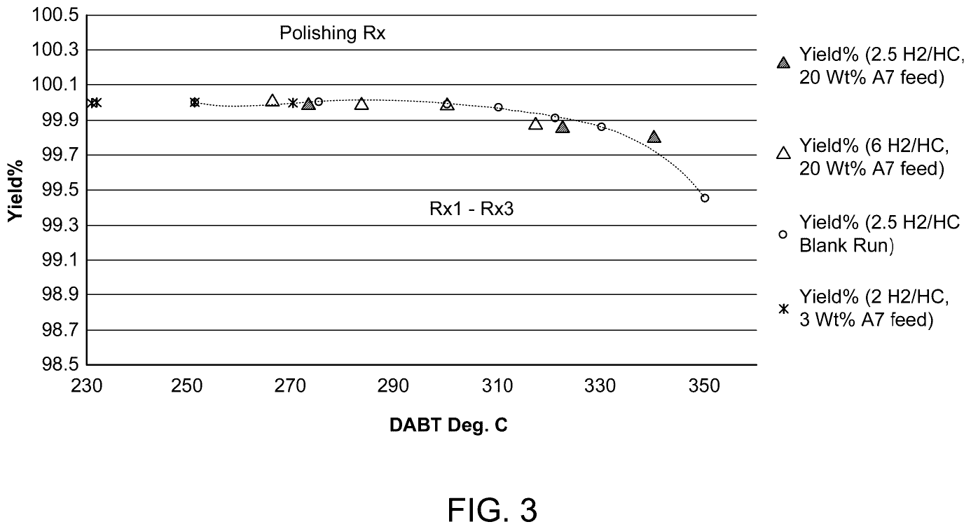

The toluene saturation process was demonstrated in an experimental pilot plant with ⅞″ downflow isothermal reactors filled with a highly selective hydrogenation catalyst. The experimental pilot plant consisted of two feed systems-Feed A (Toluene+MCH) and Feed B (Hydrogen). The temperature and pressure of the reactor was maintained so as to simulate an individual reactor in a commercial process. Two feed cases were demonstrated-Toluene 20% (balance MCH) simulating feed composition of Reactor 1, Reactor 2 or intermediate Rx and Toluene 3% (balance MCH) simulating the feed composition of the polishing reactor. Various liquid hourly space velocities ranging between 8-25 and H₂/HC ranging between 2-6 were tested. Single pass yields in a reactor can be observed from FIG. 3. The dotted line in FIG. 3 represents the impact of wall or diluent of the experimental pilot plant. Without wall effects, the selectivity is expected to be higher.

### Example 2—Dehydrogenation

The methylcyclohexane dehydrogenation (MCH) process was demonstrated in an experimental pilot plant with a ⅞″ downflow reactor filled with a highly selective dehydrogenation catalyst. The reactor was encased in a furnace. The experimental pilot plant consisted of two feed systems-MCH (99.8% purity) and hydrogen. The reactor was maintained at 80 psig, LHSV=4, H₂:HC molar ratio of 4, and a reactor temperature of approximately 400° C. The results are shown after 600 hours on stream, at which point performance is stable.

| | |
|---|---|
| MCH Conversion | 98% |
| Reaction Selectivity | >99.9% |
| MCH →Toluene + H2 | |

### SPECIFIC EMBODIMENTS

While the following is described in conjunction with specific embodiments, it will be understood that this description is intended to illustrate and not limit the scope of the preceding description and the appended claims.

A first embodiment of the invention is a process for transporting hydrogen by saturating toluene to methylcyclohexane comprising splitting a hydrocarbon feed stream comprising toluene into at least first and second hydrocarbon streams; saturating at least a portion of the toluene in the first hydrocarbon stream in a first hydrogenation reactor of a hydrogenation reaction section comprising at least one hydrogenation reactor in the presence of hydrogen and a first hydrogenation catalyst having very high methyl cyclohexane selectivity to produce a first saturated effluent stream comprising methylcyclohexane, the first hydrogenation reactor operating in vapor phase; passing the first saturated effluent stream and the second hydrocarbon stream to a second hydrogenation reactor in the hydrogenation reaction section in the presence of hydrogen and a second hydrogenation catalyst to produce a second saturated effluent stream comprising additional methylcyclohexane, the second hydrogenation reactor operating in vapor phase; passing the second saturated effluent stream to a polishing reactor in the presence of a polishing hydrogenation catalyst to produce a polishing reactor effluent stream, the polishing reactor operating in vapor phase; separating the polishing reactor effluent stream in a high pressure separator into a liquid stream comprising the methylcyclohexane and a vapor stream comprising hydrogen, wherein the vapor stream comprises at least a portion of the hydrogen in the first or second hydrogenation reactors; splitting the liquid stream into an optional recycle hydrocarbon stream and a second liquid stream; optionally recycling the recycle hydrocarbon stream to the hydrocarbon feed stream, the first hydrocarbon stream, the second hydrocarbon stream, the first hydrogenation reactor, the second hydrogenation reactor, or combinations thereof; stabilizing the second liquid stream to form a product stream comprising methylcyclohexane; and transporting the product stream to a second location. An embodiment of the invention is one, any or all of prior embodiments in this paragraph up through the first embodiment in this paragraph further comprising passing a portion of the vapor stream through a membrane or a sponge oil system to remove byproducts to form a hydrogen rich stream and a reject stream; compressing the hydrogen rich stream and sending the compressed hydrogen rich stream to the hydrocarbon feed stream, first hydrocarbon stream, the second hydrocarbon stream, the first hydrogenation reactor, the second hydrogenation reactor, or combinations thereof. An embodiment of the invention is one, any or all of prior embodiments in this paragraph up through the first embodiment in this paragraph wherein 0.01-35% of the vapor stream is passed through the membrane or the sponge oil system. An embodiment of the invention is one, any or all of prior embodiments in this paragraph up through the first embodiment in this paragraph wherein the hydrocarbon feed stream is pre-heated using heat of reaction from the first hydrogenation reactor, the second hydrogenation reactor, or both. An embodiment of the invention is one, any or all of prior embodiments in this paragraph up through the first embodiment in this paragraph further comprising superheating a steam stream generated from heat of reaction of one of the hydrogenation reactors using heat of reaction of another hydrogenation reactor. An embodiment of the invention is one, any or all of prior embodiments in this paragraph up through the first embodiment in this paragraph further comprising passing the second liquid stream through a pressure recovery turbine before stabilizing the second liquid stream to generate power and utilizing the power in the process or exporting the power to a second process. An embodiment of the invention is one, any or all of prior embodiments in this paragraph up through the first embodiment in this paragraph further comprising generating power from the superheated steam stream. An embodiment of the invention is one, any or all of prior embodiments in this paragraph up through the first embodiment in this paragraph wherein the steam stream has a pressure in the range of 350 kPa (g) to 2500 kPa (g) (3.5 barg to 25 barg). An embodiment of the invention is one, any or all of prior embodiments in this paragraph up through the first embodiment in this paragraph further comprising pre-treating the hydrocarbon feed stream, the hydrogen, or both in an adsorbent bed to remove contaminants. An embodiment of the invention is one, any or all of prior embodiments in this paragraph up through the first embodiment in this paragraph further comprising recycling part of an off-gas stream from a stabilizer overhead stream to a make-up gas compressor suction. An embodiment of the invention is one, any or all of prior embodiments in this paragraph up through the first embodiment in this paragraph wherein the first hydrogenation catalyst or the second hydrogenation catalyst or both comprises a metal of Group VIII of the Periodic Table and optionally a metal of Group I of the Periodic Table. An embodiment of the invention is one, any or all of prior embodiments in this paragraph up through the first embodiment in this paragraph wherein the first hydrogenation catalyst or the second hydrogenation catalyst or both comprises 0.05 wt % to 30 wt % of a metal of Group VIII of the Periodic Table and optionally 0.1 wt % to 3 wt % of a metal of Group I of the Periodic Table. An embodiment of the invention is one, any or all of prior embodiments in this paragraph up through the first embodiment in this paragraph wherein the hydrogenation reaction section further comprises an intermediate hydrogenation reactor between the first and second hydrogenation reactors, the process further comprising splitting the hydrocarbon feed stream comprising toluene into at least the first and second hydrocarbon streams and a third hydrocarbon stream; before passing the second hydrocarbon stream to the second hydrogenation reactor, passing the first saturated effluent stream and the third hydrocarbon stream to the intermediate hydrogenation reactor in the presence of hydrogen and an intermediate hydrogenation catalyst to form an intermediate saturated effluent stream comprising additional methylcyclohexane, the intermediate hydrogenation reactor operating in the vapor phase; and passing the intermediate saturated effluent stream to the second hydrogenation reactor. An embodiment of the invention is one, any or all of prior embodiments in this paragraph up through the first embodiment in this paragraph where the polishing reactor has an inlet temperature at least 20° C. higher than a dew point of the second or intermediate reactor effluent stream.

A second embodiment of the invention is a process for transporting hydrogen by saturating toluene to methylcyclohexane comprising splitting a hydrocarbon feed stream comprising toluene into first, intermediate, and second hydrocarbon streams; saturating at least a portion of the toluene in the first hydrocarbon stream in a first hydrogenation reactor of a hydrogenation reaction section comprising at least a first hydrogenation reactor, an intermediate hydrogenation reactor, and a second hydrogenation reactor in the presence of hydrogen and a first hydrogenation catalyst having very high methyl cyclohexane selectivity to produce a first saturated effluent stream comprising methylcyclohexane, the first hydrogenation reactor operating in vapor phase; passing the first saturated effluent stream and the intermediate hydrocarbon stream to the intermediate hydrogenation reactor in the presence of hydrogen and an intermediate hydrogenation catalyst to produce an intermediate saturated effluent stream comprising additional methylcyclohexane, the intermediate hydrogenation reactor operating in vapor phase; passing the intermediate saturated effluent stream and the second hydrocarbon stream to the second hydrogenation reactor in the presence of hydrogen and a second hydrogenation catalyst to produce a second saturated effluent stream comprising additional methylcyclohexane, the second hydrogenation reactor operating in vapor phase; passing the second saturated effluent stream to a polishing reactor in the presence of a polishing hydrogenation catalyst to produce a polishing reactor effluent stream, the polishing reactor operating in vapor phase; separating the polishing reactor effluent stream in a high pressure separator into a liquid stream comprising the methylcyclohexane and a vapor stream comprising hydrogen, wherein the vapor stream comprises at least a portion of the hydrogen in the first, intermediate, or second hydrogenation reactors; splitting the liquid stream into a recycle hydrocarbon stream and a second liquid stream; recycling the recycle hydrocarbon stream to the hydrocarbon feed stream, the first hydrocarbon stream, the intermediate hydrocarbon stream, the second hydrocarbon stream, the first hydrogenation reactor, the intermediate hydrogenation reactor, the second hydrogenation reactor, or combinations thereof; stabilizing the second liquid stream to form a product stream comprising methylcyclohexane; pre-heating the hydrocarbon feed stream using heat of reaction of the first hydrogenation reactor, the intermediate hydrogenation reactor, the second hydrogenation reactor, or combinations thereof; superheating a steam stream generated from the heat of reaction of one of the hydrogenation reactors using a heat of reaction of another hydrogenation reactor; and transporting the product stream to a second location. An embodiment of the invention is one, any or all of prior embodiments in this paragraph up through the second embodiment in this paragraph further comprising passing a portion of the vapor stream through a membrane or a sponge oil system to remove light ends to form a hydrogen rich stream and a reject stream; compressing the hydrogen rich permeate and sending the compressed hydrogen rich stream to the hydrocarbon feed stream, the first hydrocarbon stream, the intermediate hydrocarbon stream, the second hydrocarbon stream, the first hydrogenation reactor, the intermediate reactor, the second hydrogenation reactor, or combinations thereof.

A third embodiment of the invention is a process for transporting hydrogen by saturating toluene to methylcyclohexane comprising splitting a hydrocarbon feed stream comprising toluene into at least first and second hydrocarbon streams; saturating at least a portion of the toluene in the first hydrocarbon stream in a first hydrogenation reactor of a hydrogenation reaction section comprising at least one hydrogenation reactor in the presence of hydrogen and a first hydrogenation catalyst having very high methyl cyclohexane selectivity to produce a first saturated effluent stream comprising methylcyclohexane, the first hydrogenation reactor operating in vapor phase; passing the first saturated effluent stream and the second hydrocarbon stream to a second hydrogenation reactor in the hydrogenation reaction section in the presence of hydrogen and a second hydrogenation catalyst to produce a second saturated effluent stream comprising additional methylcyclohexane, the second hydrogenation reactor operating in vapor phase; passing the second saturated effluent stream to a polishing reactor in the presence of a polishing hydrogenation catalyst to produce a polishing reactor effluent stream, the polishing reactor operating in vapor phase; separating the polishing reactor effluent stream in a high pressure separator into a liquid stream comprising the methylcyclohexane and a vapor stream comprising hydrogen, wherein the vapor stream comprises at least a portion of the hydrogen in the first or second hydrogenation reactors; splitting the liquid stream into an optional recycle hydrocarbon stream and a second liquid stream; optionally recycling the recycle hydrocarbon stream to the hydrocarbon feed stream, the first hydrocarbon stream, the second hydrocarbon stream, the first hydrogenation reactor, the second hydrogenation reactor, or combinations thereof; stabilizing the second liquid stream to form a product stream comprising methylcyclohexane; transporting the product stream to a second location; dehydrogenating the methylcyclohexane in the product stream in a dehydrogenation reaction section comprising one or more dehydrogenation reactors in the presence of a dehydrogenation catalyst to form a toluene stream comprising toluene and a hydrogen product stream comprising hydrogen; passing the toluene stream to a stabilizer column to recover a toluene product stream; and recovering the hydrogen product stream. An embodiment of the invention is one, any or all of prior embodiments in this paragraph up through the third embodiment in this paragraph wherein the dehydrogenation catalyst comprises Al₂O₃, a noble metal, and an alkali or alkaline earth metal to achieve a selectivity in the dehydrogenation of methylcyclohexane to toluene and hydrogen in excess of 99.8%. An embodiment of the invention is one, any or all of prior embodiments in this paragraph up through the third embodiment in this paragraph further comprising passing the dehydrogenated product into a gas/liquid separator followed by stabilization of the separator liquid stream and passing the stabilized product through a deisoheptanizer column for further fractionation into a C6/C7 stream comprising hexane and heptane, the recycle methylcyclohexane stream and the toluene product stream, where in the toluene product stream can be shipped back to the hydrogenation unit. An embodiment of the invention is one, any or all of prior embodiments in this paragraph up through the third embodiment in this paragraph further comprising splitting the separator gas stream into a recycle hydrogen stream and a product hydrogen stream for further compression and distribution.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A process for transporting hydrogen by saturating toluene to methylcyclohexane comprising:
splitting a hydrocarbon feed stream comprising toluene into at least first and second hydrocarbon streams;
saturating at least a portion of the toluene in the first hydrocarbon stream in a first hydrogenation reactor of a hydrogenation reaction section comprising at least one hydrogenation reactor in the presence of hydrogen and a first hydrogenation catalyst having very high methyl cyclohexane selectivity to produce a first saturated effluent stream comprising methylcyclohexane, the first hydrogenation reactor operating in vapor phase;
passing the first saturated effluent stream and the second hydrocarbon stream to a second hydrogenation reactor in the hydrogenation reaction section in the presence of hydrogen and a second hydrogenation catalyst to produce a second saturated effluent stream comprising additional methylcyclohexane, the second hydrogenation reactor operating in vapor phase;
passing the second saturated effluent stream to a polishing reactor in the presence of a polishing hydrogenation catalyst to produce a polishing reactor effluent stream, the polishing reactor operating in vapor phase;
separating the polishing reactor effluent stream in a high pressure separator into a liquid stream comprising the methylcyclohexane and a vapor stream comprising hydrogen, wherein the vapor stream comprises at least a portion of the hydrogen in the first or second hydrogenation reactors;
splitting the liquid stream into an optional recycle hydrocarbon stream and a second liquid stream;
optionally recycling the recycle hydrocarbon stream to the hydrocarbon feed stream, the first hydrocarbon stream, the second hydrocarbon stream, the first hydrogenation reactor, the second hydrogenation reactor, or combinations thereof;
stabilizing the second liquid stream to form a product stream comprising methylcyclohexane; and
transporting the product stream to a second location.

2. The process of claim 1 further comprising:
passing a portion of the vapor stream through a membrane or a sponge oil system to remove light ends to form a hydrogen rich stream and a reject stream; and
compressing the hydrogen rich stream and sending the compressed hydrogen rich stream to the hydrocarbon feed stream, first hydrocarbon stream, the second hydrocarbon stream, the first hydrogenation reactor, the second hydrogenation reactor, or combinations thereof.

3. The process of claim 2 wherein 0.01-35% of the vapor stream is passed through the membrane or the sponge oil system.

4. The process of claim 1 wherein the hydrocarbon feed stream is pre-heated using heat of reaction from the first hydrogenation reactor, the second hydrogenation reactor, or both.

5. The process of claim 1 further comprising:
superheating a steam stream generated from heat of reaction of one of the hydrogenation reactors using heat of reaction of another hydrogenation reactor.

6. The process of claim 1 further comprising:
passing the second liquid stream through a pressure recovery turbine before stabilizing the second liquid stream to generate power and utilizing the power in the process or exporting the power to a second process.

7. The process of claim 5 further comprising:
generating power from the superheated steam stream.

8. The process of claim 5 wherein the steam stream has a pressure in the range of 350 kPa (g) to 2500 kPa (g) (3.5 barg to 25 barg).

9. The process of claim 1 further comprising:
pre-treating the hydrocarbon feed stream, the hydrogen, or both in an adsorbent bed to remove contaminants.

10. The process of claim 1 further comprising:
recycling part of an off-gas stream from a stabilizer overhead stream to a make-up gas compressor suction.

11. The process of claim 1 wherein the first hydrogenation catalyst or the second hydrogenation catalyst or both comprises a metal of Group VIII of the Periodic Table and optionally a metal of Group I of the Periodic Table.

12. The process of claim 1 wherein the first hydrogenation catalyst or the second hydrogenation catalyst or both comprises 0.05 wt % to 30 wt % of a metal of Group VIII of the Periodic Table and optionally 0.1 wt % to 3 wt % of a metal of Group I of the Periodic Table.

13. The process of claim 1 wherein the hydrogenation reaction section further comprises an intermediate hydrogenation reactor between the first and second hydrogenation reactors, the process further comprising:
splitting the hydrocarbon feed stream comprising toluene into at least the first and second hydrocarbon streams and a third hydrocarbon stream;
before passing the second hydrocarbon stream to the second hydrogenation reactor, passing the first saturated effluent stream and the third hydrocarbon stream to the intermediate hydrogenation reactor in the presence of hydrogen and an intermediate hydrogenation catalyst to form an intermediate saturated effluent stream comprising additional methylcyclohexane, the intermediate hydrogenation reactor operating in the vapor phase; and
passing the intermediate saturated effluent stream to the second hydrogenation reactor.

14. The process of claim 13 where the polishing reactor has an inlet temperature at least 20° C. higher than a dew point of the second or intermediate reactor effluent stream.

15. A process for transporting hydrogen by saturating toluene to methylcyclohexane comprising:
splitting a hydrocarbon feed stream comprising toluene into first, intermediate, and second hydrocarbon streams;
saturating at least a portion of the toluene in the first hydrocarbon stream in a first hydrogenation reactor of a hydrogenation reaction section comprising at least a first hydrogenation reactor, an intermediate hydrogenation reactor, and a second hydrogenation reactor in the presence of hydrogen and a first hydrogenation catalyst having very high methyl cyclohexane selectivity to produce a first saturated effluent stream comprising methylcyclohexane, the first hydrogenation reactor operating in vapor phase;
passing the first saturated effluent stream and the intermediate hydrocarbon stream to the intermediate hydrogenation reactor in the presence of hydrogen and an intermediate hydrogenation catalyst to produce an intermediate saturated effluent stream comprising additional methylcyclohexane, the intermediate hydrogenation reactor operating in vapor phase;
passing the intermediate saturated effluent stream and the second hydrocarbon stream to the second hydrogenation reactor in the presence of hydrogen and a second hydrogenation catalyst to produce a second saturated effluent stream comprising additional methylcyclohexane, the second hydrogenation reactor operating in vapor phase;
passing the second saturated effluent stream to a polishing reactor in the presence of a polishing hydrogenation catalyst to produce a polishing reactor effluent stream, the polishing reactor operating in vapor phase;
separating the polishing reactor effluent stream in a high pressure separator into a liquid stream comprising the methylcyclohexane and a vapor stream comprising hydrogen, wherein the vapor stream comprises at least a portion of the hydrogen in the first, intermediate, or second hydrogenation reactors;
splitting the liquid stream into a recycle hydrocarbon stream and a second liquid stream;
recycling the recycle hydrocarbon stream to the hydrocarbon feed stream, the first hydrocarbon stream, the intermediate hydrocarbon stream, the second hydrocarbon stream, the first hydrogenation reactor, the intermediate hydrogenation reactor, the second hydrogenation reactor, or combinations thereof;
stabilizing the second liquid stream to form a product stream comprising methylcyclohexane;
pre-heating the hydrocarbon feed stream using heat of reaction of the first hydrogenation reactor, the intermediate hydrogenation reactor, the second hydrogenation reactor, or combinations thereof; and
superheating a steam stream generated from the heat of reaction of one of the hydrogenation reactors using a heat of reaction of another hydrogenation reactor; and
transporting the product stream to a second location.

16. The process of claim 15 further comprising:
passing a portion of the vapor stream through a membrane or a sponge oil system to remove light ends to form a hydrogen rich stream and a reject stream; and
compressing the hydrogen rich permeate and sending the compressed hydrogen rich permeate to the hydrocarbon feed stream, the first hydrocarbon stream, the intermediate hydrocarbon stream, the second hydrocarbon stream, the first hydrogenation reactor, the intermediate reactor, the second hydrogenation reactor, or combinations thereof.

17. A process for transporting hydrogen by saturating toluene to methylcyclohexane comprising:
splitting a hydrocarbon feed stream comprising toluene into at least first and second hydrocarbon streams;
saturating at least a portion of the toluene in the first hydrocarbon stream in a first hydrogenation reactor of a hydrogenation reaction section comprising at least one hydrogenation reactor in the presence of hydrogen and a first hydrogenation catalyst having very high methyl cyclohexane selectivity to produce a first saturated effluent stream comprising methylcyclohexane, the first hydrogenation reactor operating in vapor phase;
passing the first saturated effluent stream and the second hydrocarbon stream to a second hydrogenation reactor in the hydrogenation reaction section in the presence of hydrogen and a second hydrogenation catalyst to produce a second saturated effluent stream comprising additional methylcyclohexane, the second hydrogenation reactor operating in vapor phase;
passing the second saturated effluent stream to a polishing reactor in the presence of a polishing hydrogenation catalyst to produce a polishing reactor effluent stream, the polishing reactor operating in vapor phase;
separating the polishing reactor effluent stream in a high pressure separator into a liquid stream comprising the methylcyclohexane and a vapor stream comprising hydrogen, wherein the vapor stream comprises at least a portion of the hydrogen in the first or second hydrogenation reactors;
splitting the liquid stream into an optional recycle hydrocarbon stream and a second liquid stream;
optionally recycling the recycle hydrocarbon stream to the hydrocarbon feed stream, the first hydrocarbon stream, the second hydrocarbon stream, the first hydrogenation reactor, the second hydrogenation reactor, or combinations thereof;
stabilizing the second liquid stream to form a product stream comprising methylcyclohexane;
transporting the product stream to a second location;
dehydrogenating the methylcyclohexane in the product stream in a dehydrogenation reaction section comprising one or more dehydrogenation reactors in the presence of a dehydrogenation catalyst to form a toluene stream comprising toluene and a hydrogen product stream comprising hydrogen;
passing the toluene stream to a stabilizer column to recover a toluene product stream; and
recovering the hydrogen product stream.

18. The process of claim 17 wherein the dehydrogenation catalyst comprises Al₂O₃, a noble metal, and an alkali or alkaline earth metal to achieve a selectivity in the dehydrogenation of methylcyclohexane to toluene and hydrogen in excess of 99.8%.

19. The process of claim 17 further comprising:
passing the dehydrogenated product into a gas/liquid separator followed by stabilization of the separator liquid stream and passing the stabilized product through a deisoheptanizer column for further fractionation into a C6/C7 stream comprising hexane and heptane, the recycle methylcyclohexane stream and the toluene product stream, where in the toluene product stream can be shipped back to the hydrogenation unit.

20. The process of claim 19 further comprising:
splitting the separator gas stream into a recycle hydrogen stream and a product hydrogen stream for further compression and distribution.
